(19) United States
(12) Patent Application Publication
(10) Pub. No.: US 2014/0219967 A1
(43) Pub. Date: Aug. 7, 2014

(54) Poly (3-Hydroxybutyrate/3-Hydroxyvalerate) of Lowered Crystallinity for Use in Animal Feed

(71) Applicant: Kemin Industries, Inc., Des Moines, IA (US)

(72) Inventors: Eric Peys, Balen (BE); Jan Vandenkerckhove, Testelt (BE); Johan Van hemel, Aartselaar (BE); Inge Peters, Voortkapel (Westerlo) (BE)

(73) Assignee: Kemin Industries, Inc., Des Moines, IA (US)

(21) Appl. No.: 14/141,668

(22) Filed: Dec. 27, 2013

Related U.S. Application Data
(60) Provisional application No. 61/746,448, filed on Dec. 27, 2012.

(51) Int. Cl. A61K 31/121

(52) U.S. Cl. CPC A61K 31/121
USPC 424/93.4; 514/675; 568/413; 424/94.6

(57) ABSTRACT

Animal feed conversion ratio can be increased and blood glucose levels can be decreased by feeding an efficacious amount of dispersible amorphous poly(3-hydroxybutyrate/3-hydroxyvalerate).

This application claims the benefit of U.S. Patent Application Ser. No. 61/746,448, filed Dec. 27, 2012, which is incorporated herein in its entirety by this reference.

### BACKGROUND OF THE INVENTION

The present invention relates to the use of in animal feed and particularly to the use of poly(3-hydroxybutyrate) with enhanced dispersion properties, reduced stereoregularity and hence reduced crystallinity in animal feeds. The bacterial storage polyester poly(3-hydroxybutyrate) (PHB) (FIG. 1) acts as a slow releaser of 3-hydroxybutyrate (3HB). 3-Hydroxybutyrate is known to exhibit antimicrobial and antiviral activity and plays an important role in the membrane synthesis of rapidly dividing colonic epithelial cells¹⁻⁴. As such it can be applied as a means to help maintain intestinal health and optimal epithelium condition for nutrient uptake in production animals.

PHB belongs to the family of poly(3-hydroxyalkanoates)(PHA) of which poly(3-hydroxyvaleric acid) (PHV) is considered to be the most prevalent. All bio-derived PHB is known to contain a fraction of copolymerized 3-hydroxyvaleric acid (3HV) (PHBV), sometimes referred to as poly(3-hydroxybutyrate-co-3-hydroxyvalerate) or poly(3-hydroxybutyrate/3-hydroxyvalerate).

PHA are bacterial bioplastics produced in different amounts and with variable composition depending on bacterial species and growth conditions. In industrial production the fermentation biomass may be spray-dried as such or further processed to obtain different grades of purity of PHA. Post-fermentation processing affects the physicochemical properties of the polymers. Bacterial PHA are produced and stored in membrane enclosed inclusion bodies where the desired physicochemical properties of the polymer are maintained. These membrane enclosed PHA granules exist in the amorphous state and the molecules are mobile. Upon damaging of these fragile enclosed inclusion bodies PHA start to alter their physicochemical properties by adopting ordered structures and crystallization. Hence extracellular PHA is partially crystalline with a typical degree of crystallinity around 50%-60%⁵. Physicochemical characteristics like stereoregularity including tacticity and crystallinity, surface area, dispersibility, polymer chain length and monomer composition all have an impact on the biodegradability and intestinal digestibility of these biopolymers⁵⁻⁸. By consequence it is the rate of degradation or digestion that determines the available amount of beneficial monomers, such as 3HB, during intestinal transit.

### SUMMARY OF THE INVENTION

Three different types of PHBV product were dosed into broiler feed, a purified crystalline PHB dosed at 100, 500 and 2500 g/t; a partially depolymerized, purified crystalline PHB dosed at 100 and 500 g/t; and a spray-dried fermentation biomass in which PHB was present together with its lysed producer cells dosed at 3500 g/t. These feed formulations were tested for their effect on performance expressed as blood levels of 3HB, glucose and insulin. In addition, intestinal histomorphological parameters including villus height, crypt depth and mucosa thickness were assessed. The highest dose of one of these three PHBV products significantly lowered feed conversion rate by reducing feed intake. The observed increased ratio of villus height versus crypt depth tentatively reflects a mode of action.

A second broiler trial, confirming the results with the highest dose, was set-up to study the effect of different degrees of crystallinity of PHBV as dosed into the feed on animal performance as well as on blood and histomorphological parameters. A purified crystalline PHB with a degree of crystallinity of 64% was dosed at 2500 g/t and a spray-dried fermentation biomass product containing producer cells in which PHB was present with increased dispersibility and a lower stereoregularity reflected by a degree of crystallinity of 10.5% was dosed at 700, 2100 or 3500 g/t. Their effect was checked on weight gain, feed intake; on blood levels of 3HB, glucose and insulin as well as on the intestinal histomorphological parameters; villus height, crypt depth and mucosa thickness. Both products significantly improved zootechnical performance. The more dispersible, low stereoregular/low crystallinity product had a bigger impact on ileal epithelium morphology than the less dispersible and high crystallinity product and was shown to have a significant blood glucose lowering effect.

In a third experiment, PHB was administered at 700 grams per ton. Birds fed the control diet were characterized by the lowest body weight gain (P<0.05) in comparison with the PHB treatments over the entire trial period. Birds fed the diets supplemented with the products P5 (approximately 5% crystallinity), P16 (approximately 14% crystallinity) and P24 (approximately 23% crystallinity) were characterized by a lower feed conversion ratio then the control birds (P<0.05) during the starter period. It can thus be concluded that all PHB qualities with crystallinites of 23% or less were able to improve the zootechnical performance of the birds.

### BRIEF DESCRIPTION OF THE FIGURES

FIG. 1 is a diagram of examples of poly(3-hydroxyalkanoates) (PHA).

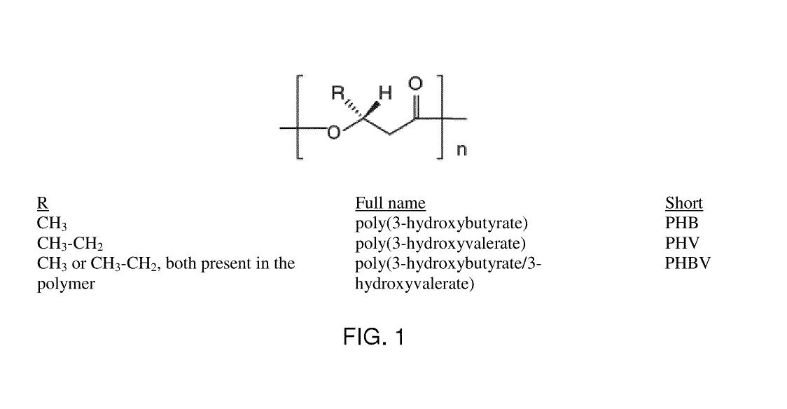

FIGS. 2A-D are light microscopic pictures of the two PHBV products: T13 with high stereoregularity (A and C) and the spray dried PHBV Y1000P with low stereoregularity, low crystallinity (B and D); observed differences relate to shape and size regularity, size distribution, surface structure, opacity, and dispersibility; each increment on the scale is represents 200 μm.

FIGS. 3A-D are light microscopic pictures of the two PHBV products: T13 with high stereoregularity (A and C) and the spraydried PHBV Y1000P with low stereoregularity, low crystallinity (B and D); each increment on the scale represents 200 μm.

### DESCRIPTION OF THE INVENTION

PHA are bacterial bioplastics produced in different amounts and with variable composition depending on bacterial species and growth conditions. In industrial production the fermentation biomass may be spray-dried as such or further processed to obtain different grades of purity of PHA. Post-fermentation processing affects the polymers' physicochemical properties. Bacterial PHA are produced and stored in membrane enclosed inclusion bodies where the desired physicochemical properties of the polymer are maintained. These membrane enclosed PHA granules exist in the amorphous state and the molecules are mobile. Upon damaging of these fragile enclosed inclusion bodies PHA start to alter their physicochemical properties by adopting ordered structures and crystallization. Hence extracellular PHA is partially crystalline with a typical degree of crystallinity around 50%-60%⁵. Physicochemical characteristics like stereoregularity including tacticity and crystallinity, surface area, dispersibility, polymer chain length and monomer composition all have an impact on the biodegradability and intestinal digestibility of these biopolymers⁵⁻⁸. By consequence it is the rate of degradation or digestion that determines the available amount of beneficial monomers, such as 3HB, during intestinal transit.

As used herein, the term “crystallinity” refers to the degree of crystallinity of a polymer and means the fraction of the ordered molecules in polymer.

As used herein, the term “dispersibility: referes to a measure of the relative extent to which something is dispersible, that is, widely distributed throughout a composition.

As used herein, the term “stereoregularity”, also known as tacticity, refers to a property that describes the regularity of the side group orientations on the backbone of a polymer. The stereoregularity of a polymer has important implications for its degree of long-range order.

As used herein, the term “amorphous” refers to a property that describes the randomness orientation of the molecules in a polymer and is in contrast to semicrystalline polymers where the molecules pack together in ordered regions. Amorphous polymers have less crystallinity than semicrystalline polymers.

### EXAMPLE 1

### Materials and Methods

PHBV products. The three PHBV products used in the presented work were obtained by fermentation of Ralstonia eutropha (TianAn Biopolymer, China). P1 is a purified PHBV (3HB at 94.2%, 3HV at 3.2%) with a number average molecular weight of 443,423 Da and a degree of crystallinity of 64%. P2 is a purified PHBV partially depolymerized by acid hydrolysis in the presence of glycerol (3HB at 81.1%, 3HV at <1%) with a number average molecular weight of 1793 Da. P3 is a fermentation biomass of which the cells were lysed and then spray-dried. P3 has a PHBV content of 60.9% (3HB 59.9%, 3HV<1%) with a number average molecular weight of 54,941 Da and a degree of crystallinity of 63.7%.

Trial setup. The trial was carried out at a facility having housing management, feeding and husbandry conditions that are regarded as representative for a modern commercial operation in Europe. The trial was carried out with 480, 1 day old male Ross 308 chicken broilers. The total treatment duration was 42 days with a 2 phase feeding system (starter, grower) (Table 1).

TABLE 1
Composition of experimental basal diets and calculated nutritional value

| | Starter | Grower |
|---|---|---|
| | Day 1-14 | Day 14-42 |
| Wheat | 41.79 | 46.43 |
| Maize | 12.0 | 17.9 |
| Soybean meal | 36.65 | 27.45 |
| Oil | 5.7 | 4.6 |
| MCP | 1.3 | 1.3 |
| Limestone | 0.4 | 0.3 |
| NaHCO₃ | 0.1 | — |
| L-Lysine | 0.56 | 0.49 |
| DL-Methionine | 0.2 | 0.18 |
| NaCl | 0.35 | 0.35 |
| Premix VIT-MIN | 1.0 | 1.0 |
| Threonine | 0.15 | — |

Calculated nutritional value

| | | |
|---|---|---|
| ME (kcal/kg) | 3054.8 | 3103.1 |
| ME (MJ/kg) | 12.8 | 13.0 |
| N × 6.25 (%) | 22.9 | 19.8 |
| Crude fibre (%) | 3.36 | 3.22 |
| Lysine (%) | 1.35 | 1.12 |
| Methionine (%) | 0.54 | 0.46 |
| Methionine + cysteine (%) | 0.93 | 0.82 |
| Ca (%) | 0.94 | 0.88 |
| P (%) | 0.42 | 0.41 |
| Na (%) | 0.20 | 0.17 |

Determined nutritional value

| | | |
|---|---|---|
| GE (kcal/kg) | 4305 | 4456 |
| N × 6.25 (%) | 23.2 | 19.3 |
| Crude fibre (%) | 3.52 | 3.19 |
| Dry matter | 89.11 | 89.01 |

The diets used in the trial were offered in mash form and were available ad libitum. Basal diets for the two experimental periods were prepared by single mixing. The animals were weighed and assigned to the treatments in order to achieve maximum possible homogeneity within each group and minimum differences between all groups.

Treatments are summarized in Table 2. One group served as a negative control receiving no treatment. Three groups were assigned to a treatment with P1 of which in-feed doses with a five-fold increase (100, 500 and 2500 g/t) were used to cover a broad range. In order to be able to compare on an equal dose basis, and because P2 was anticipated to perform better than P1, two groups were assigned to a treatment with P2 (100 and 500 g/t). One group was assigned to a treatment with P3. The intention was to use a dose of P3 equal to 2500 g/t of P1 based on PHB content, but due to limited product availability the dose was set to 3500 g/t which on PHB basis equaled 2225 g/t of P1. The experimental facility disposes of 60 floor pens for 8 broilers each. Eight pens were assigned to each of the seven treatments. Of the remaining four pens one was assigned to the control and one to each of the three PHBV products, in case of P1 and P2 to the highest dose.

TABLE 2
Treatment groups

| Group | Treatment* | Replicates | Birds per replicate |
|---|---|---|---|
| 1 | negative control | 9 | 8 |
| 2 | P1 100 g/t | 8 | 8 |
| 3 | P1 500 g/t | 8 | 8 |
| 4 | P1 2500 g/t | 9 | 8 |
| 5 | P2 100 g/t | 8 | 8 |
| 6 | P2 500 g/t | 9 | 8 |
| 7 | P3 3500 g/t | 9 | 8 |

*P1: purified crystalline PHB; P2: partially depolymerized purified crystalline PHB; P3: lysed and spray-dried fermentation biomass

Body weight gain and feed intake. Body weight was recorded at day 0, 14 and 42. Feed consumption was recorded at day 14 and 42. Both were recorded as a total per replicate (pen).

Blood parameters. Blood samples (about 5 ml) were taken at day 43 from 10 randomly selected animals per treatment group (at least 1 animal from each replicate), approximately 10 minutes after feed removal. The samples were taken from the vein under the wing and collected in tubes with clot activator. Afterwards, the blood samples were analyzed for 3HB, glucose and insulin. Blood 3HB was determined using a Pointe Scientific (Canton, MI, US) reagent set. Blood glucose level was measured using a blood glucose meter (Accu-Chek Sensor Comfort, Roche Diagnostics Corporation, Indianapolis, Iowa, US). Insulin concentrations were determined via radio-immuno-assay (RIA) using a rat insulin RIA kit (Linco Research, Millipore, Billerica, Mass., US).

Ileum histomorphological characteristics. Ileal tissue samples were taken at day 43. The samples were collected from 10 randomly selected animals per treatment group (at least 1 animal from each replicate). Segments of approximately 2 cm were taken from 2.5 cm before the ileo-caecal junction. The segments were fixed in 10% neutral buffered formalin solution and embedded in paraffin wax. Histomorphological evaluation included determination of the villus height (VH), crypt depth (CD) and mucosa thickness, according to the procedures described by Pluske et al.⁹. Measurements of mucosa thickness from villus-crypt junction to the muscularis mucosa were recorded (for ease of convenience called mucosa thickness). The data for VH and CD were determined in pairs (adjacent structures) as much as possible and only pairs were used for the VH/CD ratio calculation.

Statistics. Any statistically significant difference among experimental diets was determined by analysis of variance using the SAS software (SAS Inst. Inc., Cary, N.C., U.S.A.). Scheffé's multiple comparisons method was applied when the analysis of variance F-ratio showed a P<0.05.

### Results

Body weight gain and feed intake. Mean body weight gain, mean feed consumption and mean feed conversion ratios were calculated per replicate (pen). The replicate data were used to calculate treatment means and for analysis of variance. Data are summarized in Table 3.

TABLE 3
Broiler performance.

| Treatment | Day 0-14 | Day 14-42 | Day 0-42 |
|---|---|---|---|
| | Body weight gain (g) (mean ± SD) | | |
| negative control | 375 ± 14^{b} | 2386 ± 135^{a} | 2762 ± 140^{a} |
| P1 100 g/ton | 385 ± 10^{ab} | 2376 ± 74^{a} | 2760 ± 81^{a} |
| P1 500 g/ton | 391 ± 7^{ab} | 2342 ± 68^{a} | 2734 ± 70^{a} |
| P1 2500 g/ton | 386 ± 12^{ab} | 2387 ± 85^{a} | 2772 ± 94^{a} |
| P2 100 g/ton | 389 ± 14^{ab} | 2400 ± 129^{a} | 2789 ± 136^{a} |
| P2 500 g/ton | 398 ± 14^{a} | 2439 ± 98^{a} | 2837 ± 106^{a} |
| P3 3500 g/ton | 387 ± 15^{ab} | 2397 ± 125^{a} | 2784 ± 126^{a} |
| | Feed intake (g) (mean ± SD) | | |
| negative control | 541 ± 22^{a} | 3829 ± 210^{a} | 4369 ± 226^{a} |
| P1 100 g/ton | 551 ± 24^{a} | 3724 ± 163^{a} | 4339 ± 181^{a} |
| P1 500 g/ton | 538 ± 25^{a} | 3719 ± 200^{a} | 4257 ± 216^{a} |
| P1 2500 g/ton | 532 ± 19^{a} | 3542 ± 240^{a} | 4074 ± 248^{a} |
| P2 100 g/ton | 537 ± 24^{a} | 3802 ± 174^{a} | 4339 ± 181^{a} |
| P2 500 g/ton | 550 ± 12^{a} | 3850 ± 270^{a} | 4400 ± 265^{a} |
| P3 3500 g/ton | 531 ± 24^{a} | 3785 ± 262^{a} | 4316 ± 273^{a} |
| | Feed conversion ratio (mean ± SD) | | |
| negative control | 1.44 ± 0.05^{a} | 1.60 ± 0.04^{a} | 1.58 ± 0.04^{a} |
| P1 100 g/ton | 1.43 ± 0.07^{a} | 1.57 ± 0.05^{ab} | 1.55 ± 0.04^{ab} |
| P1 500 g/ton | 1.37 ± 0.07^{a} | 1.59 ± 0.07^{ab} | 1.56 ± 0.06^{ab} |
| P1 2500 g/ton | 1.38 ± 0.06^{a} | 1.48 ± 0.09^{b} | 1.47 ± 0.09^{b} |
| P2 100 g/ton | 1.38 ± 0.05^{a} | 1.59 ± 0.04^{ab} | 1.56 ± 0.03^{ab} |
| P2 500 g/ton | 1.38 ± 0.05^{a} | 1.58 ± 0.08^{ab} | 1.55 ± 0.07^{ab} |
| P3 3500 g/ton | 1.37 ± 0.06^{a} | 1.58 ± 0.07^{ab} | 1.55 ± 0.06^{ab} |

^{abc}means in the same column without a common letter are significantly different at P ≦ 0.05

Only during the first two weeks of the experiment, the body weight gain of the birds was significantly affected (P=0.028) by dietary treatments. The highest body weight gain was recorded when birds were fed control diet supplemented with 500 g/t of P2. Birds fed negative control diet were characterized by the lowest gain. Feed intake was never significantly affected by any of the dietary treatments but feed intake of the pens receiving P1 at 2500 g/ton seemed to be somewhat reduced during the whole treatment period. Feed conversion ratio during the grower phase and over the whole trial period was significantly lower with that same treatment when compared to the negative control.

Blood parameters. Blood samples were taken at day 43 from 10 randomly selected animals per treatment group (at least 1 animal from each replicate). Data are summarized in Table 4. P2 at 100 g/t tended to, and P2 at 500 g/t significantly raised blood glucose level (P<0.05) when compared with the negative control. Neither the level of blood 3HB nor of blood insulin was significantly affected by any of the PHB qualities. The increased blood glucose levels with P2 were accompanied by numerically higher insulin levels.

TABLE 4
Broiler blood parameters (mean ± SD) at day 43

| Treatment | Glucose (mmol/L) | 3HB (mmol/L) | Insulin (ng/mL) |
|---|---|---|---|
| negative control | 13.65 ± 0.86^{ab} | 0.460 ± 0.103^{a} | 0.870 ± 0.491^{a} |
| P1 100 g/ton | 13.70 ± 0.56^{ab} | 0.399 ± 0.057^{a} | 0.773 ± 0.245^{a} |
| P1 500 g/ton | 12.83 ± 0.91^{a} | 0.377 ± 0.113^{a} | 0.800 ± 0.475^{a} |
| P1 2500 g/ton | 13.86 ± 0.77^{ab} | 0.416 ± 0.075^{a} | 0.846 ± 0.394^{a} |
| P2 100 g/ton | 15.07 ± 1.83^{bc} | 0.406 ± 0.074^{a} | 0.950 ± 0.477^{a} |
| P2 500 g/ton | 15.63 ± 1.84^{c} | 0.373 ± 0.059^{a} | 1.016 ± 0.315^{a} |
| P3 3500 g/ton | 14.54 ± 1.37^{abc} | 0.402 ± 0.139^{a} | 0.803 ± 0.218^{a} |

^{abc}means in the same column without a common letter are significantly different at P ≦ 0.05

Ileum histomorphological characteristics. Data are summarized in Table 5. Inclusion of PHBV in the feed increased villous height when compared to the negative control group except for the low dose of P1 and for P3. Crypt depth was also affected by the treatments. P1 (500 and 2500 g/t) and P3 reduced crypt depth. The deepest crypts were observed when 100 g/t of P2 was applied. P1 at 100g/t and P3 decreased mucosa thickness, P1 at 500 and 2500 g/t had no effect, and P2 at both doses increased it compared to the negative control group. Villous height/crypt depth ratio increased significantly with 500 and 2500 g/t of P1, with 500 g/t of P2, and with 3500 g/t of P3. Treatment at 100 g/t (P1 and P2) had no effect on the villous height/crypt depth ratio.

TABLE 5
Histomorphological characteristic of broiler gastrointestinal tract
(mean ± SD).

| | | | | Villous |
|---|---|---|---|---|
| | | | Mucosa | height/crypt |
| | Villous height | Crypt depth | thickness | depth |
| Treatrment | (μm) | (μm) | (μm) | (ratio) |
| negative | 245 ± 48^{a} | 208 ± 41^{b} | 374 ± 45^{cd} | 1.24 ± 0.41^{a} |
| control | | | | |
| P1 100 g/ton | 242 ± 50^{a} | 201 ± 47^{b} | 337 ± 57^{e} | 1.26 ± 0.50^{a} |
| P1 500 g/ton | 271 ± 47^{b} | 184 ± 39^{cd} | 363 ± 45^{d} | 1.57 ± 0.47^{b} |
| P1 2500 g/ton | 281 ± 48^{bc} | 197 ± 45^{bc} | 379 ± 50^{bc} | 1.52 ± 0.46^{b} |
| P2 100 g/ton | 275 ± 51^{b} | 226 ± 55^{a} | 389 ± 57^{ab} | 1.27 ± 0.35^{a} |
| P2 500 g/ton | 291 ± 52^{c} | 202 ± 45^{b} | 399 ± 49^{a} | 1.46 ± 0.45^{b} |
| P3 3500 g/ton | 237 ± 39^{a} | 174 ± 35^{d} | 344 ± 46^{e} | 1.46 ± 0.41^{b} |

^{abc}means in the same column without a common letter are significantly different at P ≦ 0.05

### Discussion

The amount of absorbable oligo- and monomers released from a PHA polymer during intestinal transit in farm animals, thereby enabling them to exert their effects, is determined by the rate of polymer degradation during the relatively short time of passage through the digestive tract. Breakdown of the polymeric chains to monomers in the gastro-intestinal tract is probably mainly the result of both acid hydrolysis and secreted bacterial PHA depolymerases⁶. Pancreatin is also known to, albeit slowly, degrade PHB⁵. This might be explained by the fact that pancreatic lipases show the same conserved amino acid motif in their active center as PHB depolymerases¹⁰. PHB sutured to the rat stomach wall has shown to induce a tissue response by activating genes that are otherwise known to be active in the exocrine pancreas and coding for pancreatic hydrolases¹¹. Dosed into the feed, the continuous presence of PHB in the gastro-intestinal tract of broilers in this case can induce a similar response resulting in additional breakdown of polymer during transit. The PHA depolymerase exo-type enzymatic cleavage of the polymer chain i.e. a successive removal of terminal groups is known to occur at a higher rate than the endo-type enzymatic cleavage i.e. a random breakage of the polymer chain at the enzyme-binding sites. The endo-type cleavage however plays an important role at the initiation of biodegradation because it creates additional polymer chain ends accessible for the exo-type enzyme¹². Next to the properties of the PHA depolymerases involved, and the intestinal conditions they are active in, it is generally accepted that the rates of degradation are strongly influenced by the characteristics of the polymer itself, such as chemical composition, surface conditions, molecular weight and crystallinityl^{3-15.} Biodegradation of a certain amount of polymer is more effective for lower-molecular-weight polymers containing a relatively higher number of chain ends available for the exo-type degradation¹⁶. The degree of stereoregularity is also a crucial factor affecting biodegradability, since depolymerases mainly attack the amorphous domains of a polymer. The molecules in an atactic and amorphous region are loosely packed, and thus make it more prone to degradation, while crystalline parts of a polymer are more resistant^{17,18}.

The characteristics of the three PHBV products that were tested in the current broiler trial were quite diverse when it comes to those characteristics relevant to the rate of polymer degradation as described in previous paragraph. Different treatments to obtain P1 and P3 resulted in longer polymer chains for P1 with a number average molecular weight eight times that of P3 (443,423 Da and 54,941 Da for P1 and P3 respectively). Their crystallinity however was the same (64% and 63.7% respectively). P2, subjected to acid hydrolysis in the presence of glycerol, had an even lower average number molecular weight (1793 Da), being about 250 and 30 times less than that of P1 and P3 respectively. Assuming that degradation of PHBV polymers to absorbable oligo- and monomers is necessary for them to exert any effect some difference for the parameters measured during this trial was anticipated on feeding these different products to broilers. Unlike P1 and P2 the biomass product P3 contained only about 60% PHBV while the other 40% were lysed producer cells and residual fermentation broth. Any effect by these remainders can therefore not be excluded.

Broiler life performance (Table 3) during the first two weeks was significantly affected only by the highest dose of P2. This product with the lowest average number molecular weight resulted in a body weight gain that was significantly higher compared to the negative control group. With an increase in body weight gain of about 6% our results are comparable to the 5.5% increase reported by the University of Gent (Belgium) to be obtained in broiler chicks from day 8 to 22 with 1.25 kg of PHB (present as spray-dried Rhodobacter sphaeroides biomass) per ton of feed¹⁹. In that same study over the same time period a 4.7% decrease in FCR was noted. In our study there was also a trend towards an improved FCR (4.1% to 4.8%) with all but the low dose PHBV treatment of P1; the product with the highest average number molecular weight. During the final 4 weeks and considering the complete trial period the effect of P2 high dose on body weight was still there but no longer statistically significant. The life performance parameter that was affected during the final period was feed intake, which was lower (7% less) in the group treated with P1 at 2500 g/t (a statistically significant difference when compared to the negative control group) and resulted in a statistically significant reduction in FCR for this group. A lower feed intake was also observed with P1 at 500 g/t, indicating a dose response relation. These results point in the same direction as those of University of Gent who reported about 2% decrease in feed intake by broiler chickens over the period 8 to 29 days with an inclusion of 4 kg of crystalline PHB per ton of feed¹⁹.

When the energy demand is high ketone bodies can be utilized by many tissues thereby sparing glucose²⁰. Normally this glucose-sparing mechanism only becomes active under conditions of high rates of lipolysis, e.g. starvation, when sufficient amounts of ketone bodies become available in the blood. Because the concentration of blood ketone bodies in chickens is already considerably high under normal conditions they should be able to efficiently use ketone bodies for energy production²¹. As a negative feedback mechanism in order to prevent ketosis, an excess of ketone bodies is known to promote the pancreatic release of insulin. The latter inhibits lipolysis and, through the reduction of the release of fatty acids, also the production of acetyl CoA and ultimately ketone bodies²⁰. We hypothesized that if enough 3HB would be released from PHBV after oral administration and be transported to the blood via the intestinal epithelium, this could lead to a measurable difference in blood 3HB, glucose and/or insulin levels in fast growing, high energy demanding broilers. P2 was the only product that affected any of the blood parameters measured (Table 4). At the highest dose it resulted in a significantly higher blood glucose level accompanied by numerically higher blood insulin compared to the negative control group. A same trend was observed with P2 low dose. Variability of blood-3HB and blood-insulin values was high in all groups and no statistically significant differences could be detected.

Like butyrate and other short chain fatty acids B-hydroxybutyrate, the short chain fatty acid monomer of poly-B-hydroxybutyrate, was shown to have antimicrobial activity^{2,3}. Butyrate promotes performance and intestinal health of production animals by its antimicrobial potency, through its effects on cellular defense systems like the barrier function and the immune system, and through modulation of mucosal epithelial cell proliferation, differentiation and maturation²². Poly-β-hydroxybutyrate, probably through its monomer 3HB, also increases animal growth performance²³. Since butyrate and 3HB appear to form a common de novo lipid synthesis precursor pool they could possibly both exert the same trophic effect on epithelial cells^{4,24}. The length of the villi protruding in the intestinal lumen is one of the parameters that determine the surface area for nutrient absorption. Crypts can be regarded as producers of the villi. Fast tissue turnover at the villi tips needs to be compensated by rapid division of cells at the site of generation resulting in larger crypts. Increased energy and nutrient requirements for villi maintenance will lower the growth efficiency of an animal. Often the villus height/crypt depth ratio is used to quantify the combined effect of a treatment on the intestinal epithelium. The ileal histomorphological data in the current trial (Table 5) clearly show that P1 and P2 both result in an increase in villus height in a dose-related way when compared with the negative control. Villus height increased by up to 14% (P1 2500g/t) and 18% (P2 500 g/t). Any effect of P1 and P2 on crypt depth is unclear and not unambiguous. P3 did not affect villus height but clearly reduced crypt depth. Considering the villus height/crypt depth ratios all PHBV treatments resulted in an increase except for the low doses (P1 and P2 at 100 g/t). ‘Mucosa thickness’, here defined from villus-crypt junction to the muscularis mucosa, correlated to some extent (correlation coefficient 0.83) with villus height. While longer villi are associated with increased nutrient absorption and growth a thicker lamina propria is thought to be associated with less absorption and less growth²⁵. If the latter is true then the changes in villus height and villus height/ crypt depth ratio are apparently of greater importance.

In summary, at a certain level of inclusion into the feed all three PHBV products tested in this trial had a positive effect on performance. Different mechanisms seemed to be involved. P1 increased villus height and improved performance through reduction of feed intake. P2 increased villus height and blood glucose, and improved performance through increased body weight gain. P3 did not increase villus height but reduced crypt depth and ‘mucosa thickness’ and combined a reduction in feed intake with an increase in body weight gain to obtain a better performance. In all cases a reduction of tissue turnover at the villi tips, marked by an increase in villus height and or a decrease in crypt depth, appears to play a role. Whether this is due to an antimicrobial, a trophic, or an immunomodulatory effect, or a combination of these, remains undetermined. Knowing that a rapidly growing broiler devotes about 12% of newly synthesized protein, plus the necessary energy to manage and maintain this, to the digestive tract, it is clear that a reduction in tissue turnover and the accompanying changes in intestinal morphology can have a serious impact on performance²⁶. The reduction in PHBV polymer chain length as present in the current trial products and expected to favor degradation did not significantly improve animal performance parameters. A lower dosing of a possibly more effective product could have justified an extra processing cost to obtain shorter polymer chains. In a next broiler trial we will look for confirmation of the current results with P1 and we will study the effect of different degrees of dispersibility and stereoregularity including tacticity and crystallinity of PHBV on animal performance, blood and histomorphological parameters as measured in current trial.

### EXAMPLE 2

### Materials and Methods

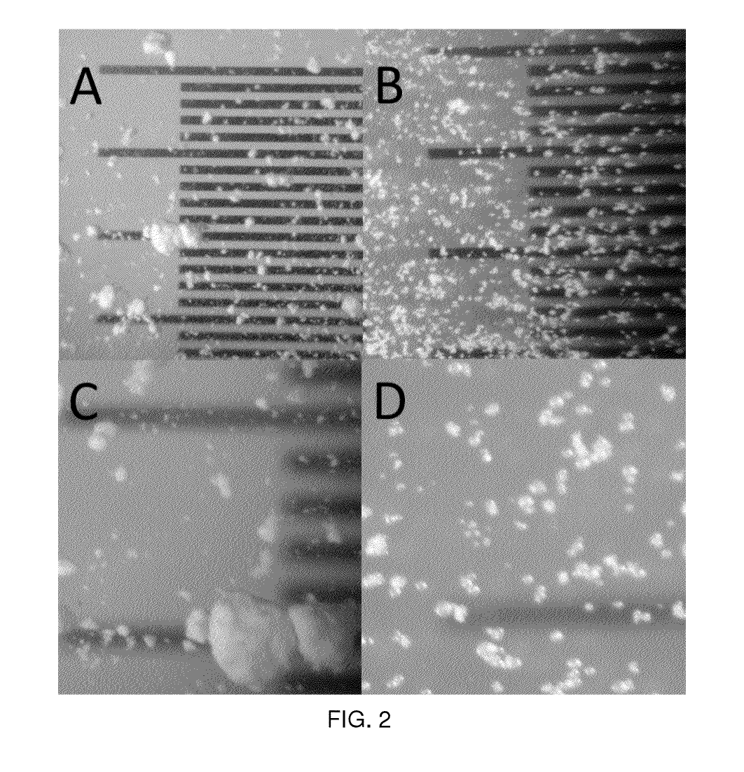

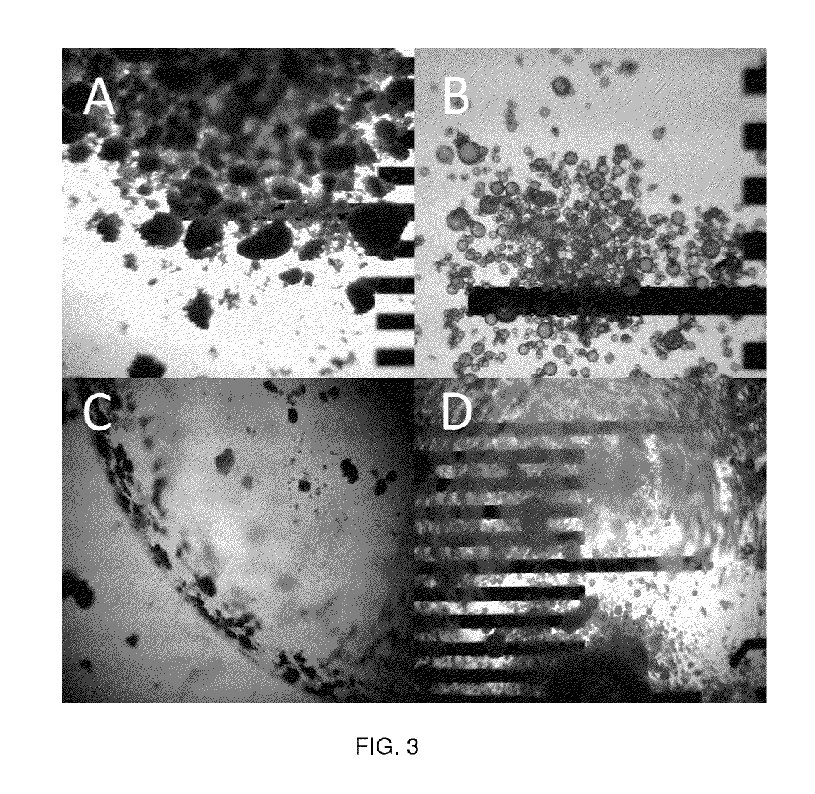

PHBV products. The two PHBV products used in current work were obtained by fermentation of Ralstonia eutropha (TianAn Biopolymer, China) (FIG. 2). T13 is a purified PHBV (3HB 94.2%, 3HV 3.2%) with a number average molecular weight of 443,423 Da and a degree of crystallinity of 64% and is actually the PHBV product with product code P1 in Example 1. Y1000P is a spray-dried fermentation biomass (3HB 71.3%, 3HV<1%) in which PHBV is still present in its producer cells with a number average molecular weight of 762,251 Da and a degree of crystallinity of 10.5%. Empirically observed differences in macroscopic properties include differences in electrostatic behavior, shape and size regularity, size distribution, surface structure, opacity, and dispersibility (FIG. 2 and FIG. 3). The detailed pictures demonstrate distinct optical and geometric different physical appearances of the two products (A and B) and the difference in dispersion properties in water (C and D). Whereas product T13 hardly distributes within the water and has a distinct tendency to agglomerate near the edges of the water droplet (C), the spray dried product readily distributes across the water volume (D).

Trial setup. The trial was carried out at a facility with the housing management, feeding and husbandry conditions that are regarded as representative for a modern commercial operation in Europe. The trial was carried out with 480, 1 day old male Ross 308 chicken broilers. The total treatment duration was 42 days with a 2 phase feeding system (starter, grower) (Table 6).

TABLE 6
Composition of experimental basal diets and calculated nutritional value

| | Starter | Grower |
|---|---|---|
| | Day 1-14 | Day 14-42 |
| Wheat | 41.68 | 45.32 |
| Maize | 12.03 | 17.9 |
| Soybean meal | 36.55 | 27.93 |
| Oil | 5.7 | 5.1 |
| MCP | 1.4 | 1.3 |
| Limestone | 0.4 | 0.3 |
| NaHCO₃ | 0.22 | 0.22 |
| L-Lysine | 0.37 | 0.33 |
| DL-Methionine | 0.3 | 0.3 |
| NaCl | 0.2 | 0.2 |
| Premix VIT-MIN | 1.0 | 1.0 |
| Threonine | 0.15 | 0.1 |

Calculated nutritional value

| | | |
|---|---|---|
| ME (kcal/kg) | 2936.2 | 3035 |
| ME (MJ/kg) | 12.3 | 12.7 |
| N × 6.25 (%) | 22.2 | 19.19 |
| Crude fibre (%) | 3.65 | 3.31 |
| Lysine (%) | 1.27 | 1.12 |
| Methionine (%) | | |
| Methionine + cysteine (%) | 0.86 | 0.80 |
| Ca (%) | 0.51 | 0.43 |
| P (%) | 0.43 | 0.40 |
| Na (%) | 0.15 | 0.15 |

Determined nutritional value

| | | |
|---|---|---|
| GE (kcal/kg) | 4287 | 4400 |
| N × 6.25 (%) | 22.7 | 19.4 |
| Crude fibre (%) | 3.50 | 3.22 |
| Dry matter | 89.2 | 88.7 |

The diets used in the trial were offered in mash form and were available ad libitum. Basal diets for the two experimental periods were prepared by single mixing. The animals were weighed and assigned to the treatments in order to achieve maximum possible homogeneity within each group and minimum differences between all groups.

Treatments are summarized in Table 7. One group served as a negative control receiving no treatment. One group served as a positive control receiving ButiPEARL™ (Kemin Industries, Inc., Des Moines, Iowa) at 200 g/t. One group was assigned to a treatment with T13 at 2500 g/t.

Three groups were assigned to a treatment with Y1000P (700, 2100 and 3500 g/t). T13 at 2500 g/t was included for confirmation of the results obtained with this product at the same dosage in Example 1 (trial product code P1)⁹. Treatments with Y1000P at 700 g/t and 3500 g/t in current trial are comparable, on a PHBV content base, to treatments with T13 at respectively 500 g/t and 2500 g/t in current or previous trial and allowed for the comparison of the effect of these two products with a different degree of PHBV crystallinity. Y1000P at 2100 g/t was included as an intermediate between 700 g/t and 3500 g/t. The experimental facility has 60 floor pens for 8 broilers each. Ten pens were assigned to each of the six treatments.

TABLE 7
Treatment groups

| Group | Treatment | | Replicates | Birds per replicate |
|---|---|---|---|---|
| 1 | negative control | | 10 | 8 |
| 2 | ButiPEARL ™ | 200 g/t | 10 | 8 |
| 3 | T13 | 2500 g/t | 10 | 8 |
| 4 | Y1000P | 700 g/t | 10 | 8 |
| 5 | Y1000P | 2100 g/t | 10 | 8 |
| 6 | Y1000P | 3500 g/t | 10 | 8 |

Body weight gain and feed intake. Body weight was recorded at day 0, 14 and 42. Feed consumption was recorded at day 14 and 42. Both were recorded as a total per replicate (pen).

Blood parameters. Blood samples (about 5 ml) were taken at day 43 from 10 randomly selected animals per treatment group (1 animal from each replicate), approximately 10 minutes after feed removal. The samples were taken from the vein under the wing and collected in tubes with clot activator. Afterwards, the blood samples were analyzed for 3HB, glucose and insulin. Blood 3HB was determined using a Pointe Scientific (Canton, Mich., US) reagent set. Blood glucose level was measured using a blood glucose meter (Accu-Chek Sensor Comfort, Roche Diagnostics Corporation, Indianapolis, Iowa, US). Insulin concentrations were determined via radio-immuno-as say (RIA) using a rat insulin RIA kit (Linco Research, Millipore, Billerica, Mass., US).

Ileum histomorphological characteristics. Ileal tissue samples were taken at day 43. The samples were collected from 10 randomly selected animals per treatment group (1 animal from each replicate). Segments of approximately 2 cm were taken from 2.5 cm before the ileo-caecal junction. The segments were fixed in 10% neutral buffered formalin solution and embedded in paraffin wax. Histomorphological evaluation included determination of the villus height (VH), crypt depth (CD) and mucosa thickness, according to the procedures described by Pluske et al.¹¹. Measurements of mucosa thickness from villus-crypt junction to the muscularis mucosa were recorded (for ease of convenience called mucosa thickness). The data for VH and CD were determined in pairs (adjacent structures) for every measurement and were used as such for VH/CD ratio calculation.

Statistics. Any statistically significant difference among experimental diets was determined by analysis of variance using STATGRAPHICS Centurion XVI software (StatPoint Technologies, Inc., Warrenton, Va., US). Duncan's multiple comparisons method was applied when the analysis of variance F-ratio showed a P<0.05 to determine which means were significantly different from which others.

### Results

Body weight gain and feed intake. Mean body weight gain, mean feed consumption and mean feed conversion ratios (mean feed consumption over mean body weight gain) were calculated per replicate (pen). The replicate data were used to calculate treatment means and for analysis of variance. Data are summarized in Table 8.

TABLE 8
Broiler performance.

| Treatment | | Day 0-14 | Day 14-42 | Day 0-42 |
|---|---|---|---|---|
| | | Body weight gain (g) (mean ± SD) | | |
| negative control | | 354 ± 31^{a} | 2402 ± 91^{a} | 2755 ± 103^{a} |
| ButiPEARL ™ | 200 g/t | 353 ± 23^{a} | 2408 ± 81^{a} | 2761 ± 90^{a} |
| T13 | 2500 g/t | 354 ± 24^{a} | 2413 ± 126^{a} | 2757 ± 137^{a} |
| Y1000P | 700 g/t | 349 ± 14^{a} | 2427 ± 117^{a} | 2775 ± 120^{a} |
| Y1000P | 2100 g/t | 364 ± 17^{a} | 2481 ± 111^{a} | 2845 ± 123^{a} |
| Y1000P | 3500 g/t | 358 ± 24^{a} | 2478 ± 112^{a} | 2836 ± 112^{a} |
| | | Feed intake (g) (mean ± SD) | | |
| negative control | | 476 ± 39^{a} | 4317 ± 189^{a} | 4793 ± 211^{a} |
| ButiPEARL ™ | 200 g/t | 475 ± 33^{a} | 4246 ± 167^{a} | 4721 ± 177^{a} |
| T13 | 2500 g/t | 458 ± 29^{a} | 4097 ± 218^{a} | 4555 ± 230^{a} |
| Y1000P | 700 g/t | 465 ± 24^{a} | 4116 ± 202^{a} | 4580 ± 203^{a} |
| Y1000P | 2100 g/t | 479 ± 21^{a} | 4244 ± 229^{a} | 4722 ± 238^{a} |
| Y1000P | 3500 g/t | 461 ± 31^{a} | 4173 ± 204^{a} | 4633 ± 218^{a} |
| | | Feed conversion ratio (mean ± SD) | | |
| negative control | | 1.35 ± 0.02^{a} | 1.80 ± 0.04^{a} | 1.74 ± 0.04^{a} |
| ButiPEARL ™ | 200 g/t | 1.34 ± 0.03^{a} | 1.76 ± 0.02^{ab} | 1.71 ± 0.02^{a} |
| T13 | 2500 g/t | 1.33 ± 0.08^{a} | 1.70 ± 0.08^{c} | 1.65 ± 0.07^{b} |
| Y1000P | 700 g/t | 1.33 ± 0.05^{a} | 1.70 ± 0.07^{c} | 1.65 ± 0.06^{b} |
| Y1000P | 2100 g/t | 1.32 ± 0.03^{a} | 1.71 ± 0.05^{bc} | 1.66 ± 0.04^{b} |
| Y1000P | 3500 g/t | 1.29 ± 0.04^{a} | 1.69 ± 0.08^{c} | 1.63 ± 0.07^{b} |

^{abc}means in the same column without a common letter are significantly different at P ≦ 0.05

Body weight gain and feed intake were never significantly affected by any of the dietary treatments. The highest body weight gain was recorded when birds were fed control diet supplemented with 2100 g/t or 3500 g/t of Y1000P. During the complete treatment period feed intake was lowest for the broilers receiving T13 at 2500 g/t. At the end of the trial period all PHBV treatment groups (T13 and Y1000P) showed significantly better FCR than the negative control group and the group treated with ButiPEARLTM at 200 g/t.

Blood parameters. Blood samples were taken at day 43 from 10 randomly selected animals per treatment group (animal from each replicate). Data are summarized in Table 9. All Y1000P treatment doses resulted in significantly lower blood glucose concentration than the negative control group as measured on day 43. Blood glucose level with T13 (2500 g/t) and ButiPEARL™ (200 g/t) not significantly differed from blood glucose level in the negative control group. Neither the level of blood 3HB nor of blood insulin was significantly affected by any of the dietary treatments.

TABLE 9
Broiler blood parameters (mean ± SD).

| Treatment | | Glucose (mmol/L) | 3HB (mmol/L) | Insulin (ng/mL) |
|---|---|---|---|---|
| negative control | | 15.02 ± 1.61^{a} | 0.445 ± 0.079^{a} | 1.208 ± 0.501^{a} |
| ButiPEARL ™ | 200 g/t | 14.85 ± 1.38^{ab} | 0.407 ± 0.100^{a} | 1.015 ± 0.258^{a} |
| T13 | 2500 g/t | 13.86 ± 1.15^{ab} | 0.407 ± 0.109^{a} | 0.967 ± 0.234^{a} |
| Y1000P | 700 g/t | 13.26 ± 1.21^{b} | 0.392 ± 0.071^{a} | 1.077 ± 0.348^{a} |
| Y1000P | 2100 g/t | 11.46 ± 1.04^{c} | 0.461 ± 0.084^{a} | 1.362 ± 0.553^{a} |
| Y1000P | 3500 g/t | 10.43 ± 0.72^{c} | 0.415 ± 0.065^{a} | 0.935 ± 0.388^{a} |

^{abc}means in the same column without a common letter are significantly different at P ≦ 0.05

Ileum histomorphological characteristics. Data are summarized in Table 10. All treatments with PHBV in the present trial increased villous height compared to the negative control group. All treatments also increased villous height/crypt depth ratio except for T13. ButiPEARL™ at 200 g/t had no effect on villous height or on villous height/crypt depth ratio, but it reduced crypt depth. Crypt depth increased with T13 and with Y1000P at 700 g/t. All treatments, including ButiPEARL™, resulted in a thicker mucosa compared to the negative control.

TABLE 10
Histomorphological characteristic of broiler gastrointestinal tract (mean ± SD)

| | | Villous | Crypt | Mucosa | |
|---|---|---|---|---|---|
| | | height | depth | thickness | Villous height/ |
| Treatment | Amount | (μm) | (μm) | (μm) | crypt depth (ratio) |
| negative | | 264 ± 67^{a} | 104 ± 17^{b} | 279 ± 72^{a} | 2.62 ± 0.80^{a} |
| control | | | | | |
| ButiPEARL ™ | 200 g/t | 256 ± 58^{a} | 101 ± 16^{a} | 296 ± 56^{b} | 2.61 ± 0.75^{a} |
| T13 | 2500 g/t | 280 ± 52^{b} | 113 ± 17^{c} | 358 ± 56^{c} | 2.53 ± 0.59^{a} |
| Y1000P | 700 g/t | 312 ± 78^{d} | 113 ± 19^{c} | 407 ± 79^{d} | 2.86 ± 0.88^{b} |
| Y1000P | 2100 g/t | 293 ± 91^{c} | 105 ± 17^{b} | 363 ± 73^{c} | 2.89 ± 1.11^{b} |
| Y1000P | 3500 g/t | 343 ± 67^{e} | 105 ± 16^{b} | 421 ± 75^{e} | 3.35 ± 0.81^{c} |

^{abc}means in the same column without a common letter are significantly different at P ≦ 0.05

### Discussion

The two PHBV products included in the current broiler trial were chosen because they are quite diverse when it comes to their physicochemical traits and degree of crystallinity of their PHBV polymer. The PHBV in Y1000P, which comprises intact dead Ralstonia eutropha containing the PHBV inside their cells, has a degree of crystallinity of 10.5%, and possess good dispersive properties in water. When compared to Y1000P, due to the purification process PHBV in T13 has a degree of crystallinity about six times higher (64%), and has low dispersive properties in water. Assuming that degradation of PHBV polymers to absorbable oligo- and monomers is necessary for them to exert any effect, and knowing that this degradation is linked to the degree of water dispersion as well as crystallinity, some difference for the parameters measured during this trial was anticipated on feeding these two products to broilers. T13 at 2500 g/t was included as a treatment for possible confirmation of the results obtained with it in Example 1. Based on an equal PHBV content Y1000P at 3500 g/t was included for comparison with T13 at 2500 g/t. On the same basis Y1000P at 700 g/t can be compared with T13 at 500 g/t in Example 1. Y1000P at 2100 g/t was chosen as intermediate. Unlike T13 the biomass product Y1000P contained only about 71-72% PHBV while the other 28-29% were producer cells and residual fermentation broth. Any effect by these remainders can therefore not be excluded. ButiPEARL™ at 200g/t was included as a positive control. ButiPEARL™ is an encapsulated source of intestinally released calcium butyrate with a low butyrate smell. It is slowly released to provide a steady source of energy for villi growth along the intestinal tract. ButiPEARL™ was included in the trial to assess a possible difference with PHB which may be important for future product positioning. Although butyric acid and 3-hydroxybutyric acid look very similar they may have a different physiological impact.

Broiler zootechnical performance (Table 3) during the first two weeks was not significantly affected by any of the treatments. As in Example 1 there was some trend towards an improved FCR with all PHBV treatments but it was less clear. The reduction in FCR was best observed with the highest dose of Y1000P, and with about 4% percent comparable to the 4.1% to 4.8% observed in Example 1 with different PHBV products and to the 4.7% decrease reported by the University of Gent (Belgium) obtained in broiler chicks from day 8 to 22 with 1.25 kg of PHB (present as spray-dried Rhodobacter sphaeroides biomass) per ton of feed¹⁹. Considering the complete trial period all PHBV treated groups showed a significantly better FCR than both the negative control and the ButiPEARL™ at 200g/t group. As in Example 1, T13 at 2500 g/t significantly affected feed intake when compared only to the negative control group (T-test). Feed intake with T13 at 2500 g/t was 5.2% and 7% lower in respectively current trial and Example 1 when compared to the respective negative control groups, while body weight gain was the same. These results point in the same direction as those of University of Gent who reported about 2% decrease in feed intake by broiler chickens over the period 8 to 29 days with an inclusion of 4 kg of crystalline PHB per ton of feed¹⁹.

As in Example 1, T13 did not affect any of these blood parameters. All Y1000P doses resulted in a statistically significant reduction of blood glucose level. An 11.7%, 23.7% and 30.6% reduction at doses of respectively 700 g/t, 2100 g/t and 3500 g/t indicates some kind of dose-response relation. Variability of blood-3HB and blood-insulin values was again high in all groups and no statistically significant differences could be detected.

The ileal histomorphological data of current trial (Table 10) clearly show that T13, as in Example 1, and Y1000P both result in an increase in villus height when compared with the negative control. Villus height increased by 6% (T13 2500 g/t, was 14% in Example 1) and by 18.2%, 11% and 29.9% (Y1000P at 700 g/t, 2100 g/t and 3500 g/t respectively). ButiPEARL™ not affected villous height but was the only treatment that reduced crypt depth in the current trial. Crypts were significantly deeper with T13 (no effect in previous trial) and with Y1000P at 700 g/t (both 8.7%). While villous heights were comparable, crypts were significantly less deep in current trial than in Example 1 (about a factor 2 difference). As a result the villous height/crypt depth ratios in current trial were about double those of Example 1. Considering the villus height/crypt depth ratio all Y1000P doses resulted in an increase of this ratio, with 9.2%, 10.3% and 27.9% for 700 g/t, 2100 g/t and 3500 g/t respectively compared to the negative control group. Mucosa thickness, here defined from villus-crypt junction to the muscularis mucosa, again correlated well with villus height (correlation coefficient 0.93, was 0.83 in Example 1).

Based on the Example 1 results we concluded previously that an increase in villous height and/or villous height/crypt depth ratio was probably a marker of the efficiency of a PHBV product to improve broiler performance. A reduction of tissue turnover at the villi tips, marked by an increase in villus height and or a decrease in crypt depth, appeared to accompany improved performance. Knowing that a rapidly growing broiler devotes about 12% of newly synthesized protein, plus the necessary energy to manage and maintain this, to the digestive tract, it is clear that a reduction in tissue turnover can have a serious impact on performance25. Additionally, increased villous height might be related to a higher absorptive intestinal surface which might facilitate nutrient absorption. Current results with T13 at 2500 g/t confirm these of Example 1. The increase in villous height with T13 at 2500 g/t in current trial was accompanied by deeper crypts resulting in their ratio not being different from that of the negative control nor from that of ButiPEARL™ at 200 g/t. Zootechnical performance however had clearly improved with comparable body weight gain and FCR nine points lower than that of the negative control group. Both T13 and Y1000P at all doses included significantly improved zootechnical performance (P<0.05) compared to the negative control group. The different degree of crystallinity of their PHBV polymer did not result in a statistically significant performance difference when products were dosed at equal PHBV amounts (T13 at 2500 g/t and Y1000P at 3500 g/t). Blood glucose level was significantly lower in the group treated with the PHBV polymer that was less crystalline (Y1000P) (t-test, P<0.000001). Villous height, villous height/crypt depth ratio and mucosa thickness were all significantly larger with Y1000P, while crypt depth was significantly less.

It might be that, comparable to dietary supplementation of organic acids, dietary supplementation of biodegradable PHBV in broiler chickens not only favors a healthy intestinal epithelium in optimal condition for nutrient uptake but also results in a kind of growth stimulation of the small intestine as a whole27,28. Upcoming trials may not only consider the microscopic changes at the intestinal epithelial surface but also the macroscopic changes in size of the small intestine. The blood glucose lowering effect, with dosed response, observed with Y1000P in present trial is also worth looking into. A valuable explanation for the observations made in current trial could be a change in the metabolic rate or of certain hormone levels potent to regulate nutritional metabolism. Indeed, the observed blood glucose lowering effect could point at an effect at the endocrine level. Such changes could be due to e.g. an increase in the GLP-1 (glucagon-like peptide-1), which is known to have an effect on blood glucose levels. Furthermore, GLP-2 (glucagon-like peptide-2) which is co-secreted with GLP-1 (e.g. from intestinal L cells) is known to increase intestinal proliferation, which could be an explanation for the observed improvement in ileal histomorphology in the broiler trials of Examples 1 and 2. The increased concentration of GLP-1 (and GLP-2) could be due to a higher production of these hormones or to a reduction of DPP-4 (dipeptidyl peptidase 4). GLP-1 and GLP-2 are rapidly inactivated by the enzyme dipeptidyl peptidase-4 (DPP-4). The effects on GLP levels could be due to a direct activity of PHB/PHBV, or via an indirect action (e.g. a prebiotic effect of PHB/PHBV resulting in an increase in particular gastro-intestinal bacteria that increase production of GLP-1 and GLP-2)

As such, PHB/PHBV cannot only find use in increasing animal performance, but also have human applications, such as the treatment of diabetes and intestinal diseases such as, but not limited to, inflammatory bowel diseases (IBD), colon cancer, short bowel syndrome (SBS), and chemotherapy-induced apoptosis.

### EXAMPLE 3

### SUMMARY

The aim of the study was to determine the effect of different qualities of product P on the growth performance of broilers.

The trial was carried out with 480, 1 day old male Ross 308 chicken broilers in 5 treatments (1—control treatment, 2—control+P5, 3—control+P14, 4—control+P16 and 5—control+P24) with 12 replicates per treatment and 8 birds per replicate. The total treatment duration was 42 days with a 2 phase feeding system (starter 1-14d), grower (14-42d).

Birds fed control diet were characterized by the lowest BWG (p<0.05) in comparison with experimental treatments. Birds fed diets supplemented with product P5, P16 and P24 were characterized by lower FCR then control birds (p<0.05). Throughout the grower period tendencies (p<0.1) to improved FCR after product P supplementation were noted. All experimental treatments were characterized by lower FCR than control treatment.

### Study Objective

A broiler trial was conducted in order to study the effect of different qualities of product P on the growth performance.

### Materials and Methods

### Husbandry Conditions

The trial was carried out at a contract facility. The housing management, feeding and husbandry conditions used in the facility were regarded as representative for a modern commercial operation in Europe. This trial was carried out with 480, 1 day old male Ross 308 chicken broilers in 5 treatments with 12 replicates per treatment and 8 birds per replicate. The total treatment duration was 42 days with a 2 phase feeding system (starter 1-14d), grower (14-42d)

The animals were weighed, selected out of groups coming from a breeding farm without significant disease problem, and assigned to the treatments in order to achieve maximum possible homogeneity within each group and minimum differences between all trial groups. Six groups were set up according to the scheme of Table 11.

TABLE 11
Treatments scheme

| | | Degree of | |
|---|---|---|---|
| Treatment # | Treatment name | crystallinity | Dose |
| 1 | control | | — |
| 2 | Product P5 | 5% | 1400 g/ton |
| 3 | Product P14 | 14% | 1400 g/ton |
| 4 | Product P16 | 14% | 1400 g/ton |
| 5 | Product P24 | 23% | 1400 g/ton |

### Diet Formulation

Feeds were manufactured at the facility. The trial was divided into two feeding periods—starter (0-14d), grower (14-42d). The composition of experimental diets and calculated value is presented in Table 12. The diets used in the trial were offered in mash form and available ad libitum. Neither growth promoters were used in the diets. The final feeds were manufactured in horizontal mixers −100 or 300 kg per batch, model: Zuptor 100 or 300 MPW, mixing time 4-6 minutes, mixing band: 27.4-28.8 rev/min.

### Analysis Relating to Diet

The concentration of dry matter, crude protein, and crude fiber in diets, were determined using the AOAC procedure (AOAC, 2005). Nitrogen content was analyzed by the Kjel Foss Automatic 16210 (A/S N. Foss Electric, Denmark). Fat content was determined using the Soxtec System HT 1043, Extraction Unit (Foss Tecator, Denmark). Gross energy was determined using an adiabatic bomb calorimeter (KL 12Mn, Precyzja-Bit PPHU, Poland) standardized with benzoic acid.

TABLE 12
Composition of experimental basal diets and
calculated nutritional value (0-14 d, 14-42 d)

| | Starter | Grower |
|---|---|---|
| | Day 1-14 | Day 14-42 |
| Wheat (CP 15.6%) | 44.547 | 45.023 |
| Maize (CP 9.4%) | 10 | 17.286 |
| Soybean meal (CP 44%) | 34.543 | 27.67 |
| Oil | 6.326 | 6.228 |
| MCP | 1.679 | 1.305 |
| Limestone | 0.708 | 0.388 |
| NaHCO₃ | 0.300 | 0.300 |
| L-Lys | 0.303 | 0.288 |
| DL-Met | 0.321 | 0.262 |
| NaCl | 0.15 | 0.15 |
| Premix VIT-MIN (Ca 32%) | 1.0 | 1.0 |
| Thr | 0.123 | 0.099 |

Calculated nutritional value

| | | |
|---|---|---|
| ME (kcal/kg) | 2950 | 3035 |
| N × 6.25 (%) | 22.0 | 19.62 |
| Crude fiber (%) | 3.53 | 3.28 |
| Digestible AAA | | |
| Lys (%) | 1.27 | 1.1 |
| Met + cys. (%) | 0.94 | 0.84 |
| Tryptophan | 0.25 | 0.22 |
| Threonine | 0.83 | 0.73 |
| Ca (%) | 1.05 | 0.85 |
| Pₐ (%) | 0.50 | 0.42 |
| Na (%) | 0.15 | 0.15 |

Determined nutritional value n = 3, ±SD

| | | |
|---|---|---|
| Crude protein (%) | 21.77 ± 0.251 | 19.70 ± 0.092 |
| GE (kcal/kg) | 4224 ± 22.23 | 4469 ± 10.15 |
| Ca % | 1.06 ± 0.0153 | 0.85 ± 0.02 |
| Na % | 0.153 ± 0.0153 | 0.147 ± 0.006 |
| Crude fiber % | 3.69 ± 0.163 | 3.33 ± 0.064 |

### Diet Mixing and Sampling

Maize and wheat were ground using a Skiold disc mill with disc distances set at 1 mm for both wheat and maize. Tested products were mixt before use and next premixed with 10 kg wheat separately for each treatment. The experimental premixes were used for future diet preparation. Diets were mixed using a commercial horizontal feed mixer with precision 1:10000. Three 500 g samples were collected per diet—sub samples from start, middle and end of the manufacture run. Samples were blended together and 500g was sent for analysis.

### Observations During the Study

The condition of experimental animals and litter was observed twice per day.

### Body Weight Gain and Feed Intake Measurement

Body weights were recorded at day 0, 14 and 42. Feed consumption, to evaluate the feed intake (FI), was recorded at day 14 and 42.

### Environment Control

Environmental conditions (temperature maximum and minimum) and humidity were recorded daily using electronic equipment. The experimental room was equipped with two conditions recorders allocated on opposites walls. Environmental temperature in the room was modified during the trial according to the recommendations.

### Statistical Analysis of Data

Statistical differences between experimental diets were calculated by analysis of variance using the SAS software (SAS Inst. Inc., Cary, N.C., U.S.A.). Differences among diet were determined using a Duncan test comparison when the significance of the model was P<0.05 (abc). All data were previously explored to discard any possible outlier. Analysis was performed using the appropriate procedures of SAS Software (Distribution analyses; outliers were defined as observations whose distance to the location estimate exceeds 3 times standard deviation).

### Results

### Performance

During the first two weeks of the experiment, body weight gains of birds were not affected by dietary treatments (Table 13). The highest BWG was recorded when birds were fed control diet supplemented with product P5. There was tendency (p<0.1) to improve BWG after P5 product supplementation in comparison to control treatment. During the grower period (14-42) all experimental treatments were characterized by higher BWG's than control treatment, but only P14 and P24 were significantly higher (p<0.05). Considering the whole experiment (0-42nd day), birds fed control diet were characterized by the lowest BWG (p<0.05). Birds fed diet supplemented with P14 were characterized by the highest BWG's (p>0.05)

During the starter period, the grower period and the whole experiment, feed intake was not affected by dietary treatments. Total feed intake ranged from 4306 to 4341 g and the highest diet consumption was noted when birds were fed control diet (p<0.05).

Feed conversion ratio was affected during starter period by some of the tested products. Birds fed diets supplemented with product P5, P16 and P24 were characterized by lower FCR then control birds (p<0.05). Throughout the grower period tendencies (p<0.1) to improved FCR after product P supplementation were noted. All experimental treatments were characterized by lower FCR than control treatment. Considering whole experiment (0-42nd d) FCR was not significantly affected by dietary treatments but birds fed diets supplemented product P were characterized by a numercially lower FCR (p>0.05).

TABLE 13
Broiler chickens performance.

| | BWG | | | FI | | | FCR | | |
|---|---|---|---|---|---|---|---|---|---|
| | 0-14 | 14-42 | 0-42 | 0-14 | 14-42 | 0-42 | 0-14 | 14-42 | 0-42 |
| control | 402 | 2220^{b} | 2616^{b} | 450 | 3891 | 4341 | 1.14a | 1.73 | 1.64 |
| Product P5 | 425 | 2285^{ab} | 2709^{a} | 459 | 3851 | 4315 | 1.08b | 1.70 | 1.59 |
| Product P14 | 415 | 2331^{a} | 2750^{a} | 460 | 3845 | 4306 | 1.10ab | 1.66 | 1.58 |
| Product P16 | 411 | 2293^{ab} | 2704^{a} | 425 | 3901 | 4339 | 1.06b | 1.70 | 1.60 |
| Product P24 | 407 | 2320^{a} | 2733^{a} | 444 | 3905 | 4339 | 1.06b | 1.69 | 1.60 |
| SEM | 2.637 | 11.84 | 13.16 | 4.349 | 16.33 | 18.37 | 0.0084 | 0.0088 | 0.0085 |
| P | 0.0755 | 0.0262 | 0.0100 | 0.0936 | 0.7032 | 0.9663 | 0.016 | 0.0885 | 0.1337 |

SEM—standard error of the mean
P—probability
^{abc}means in the same column without a common letter are significantly different at p ≦ 0.05

### Health and Condition

The consistency of the feces during the whole trial was normal. No sticky droppings or diarrhea problems were observed. There were no differences between dietary treatments. Litter quality was similar across treatments during whole trial. There was mortality during the experiment but not related to experimental treatments.

### Discussion

In Example 1, a significant improvement in broiler performance (FCR) was observed in the group administered PHB product P1 (T13) at a dosage of 2.5 kg/ton. At lower dosages this product was not effective. Product P3, consisting of a fermentation broth spray-dried after cell lysis, did not show an effect on the FCR in this experiment at 3.5 kg/ton (2.5 kg/ton PHB). This was rather unexpected, since the particle size of the PHB granules was significantly smaller in product P3 compared to product P1. Forni et al⁸ suggested that reducing the average particle size results in greater digestibility.

Halet et al. conducted experiments for the protection of Artemia franciscana against pathogenic Vibrio bacteria. They observed that PHB-containing bacteria were effective at a 100× lower dosage than the PHB powder, which the authors attributed to their smaller particle size²⁹. However, reducing the PHB particle size in Example 1 did not lead to a better bioavailability and thus animal performance.

In Example 2, the same PHB product T13 was used at 2.5 kg/ton as in Example 1. Again, this resulted in a significant improvement of the FCR compared to the control group as observed in Example 1. When product Y1000P, a spray-dried fermentation broth, was added to the feed, the FCR significantly improved in the groups administered 0.7, 2.1 and 3.5 kg/ton (containing 0.5, 1.5 and 2.5 kg/ton PHB respectively). Using this product, animal performance could be improved at dosages as low as 0.7 kg/ton, which was not possible with the T13 product. Moreover, at 3.5 kg/ton the Y1000P PHB is effective, whereas the P3 product was not at this dosage in Example 1.

Since the tests have shown that there is no significant difference in the average size of the PHB particles in PHB products P3 and Y1000P, it can be concluded that the smaller particle size is not responsible for the improvement of the in vivo effect as suggested by Halet et al. Since the only difference between PHB products P3 and Y1000P is the crystallinity of the PHB polymer (64% vs. 10.5% respectively), it can be concluded that the use of more amorphous PHB results in improved animal performances. Lowering the crystallinity of the PHB polymer allows the administration of lower PHB dosages in the feed without compromising the desired effects.

In Example 3, PHB was administered at 700 grams per ton. Birds fed the control diet were characterized by the lowest body weight gain (P<0.05) in comparison with the PHB treatments over the entire trial period. Birds fed the diets supplemented with the products P5 (approximately 5% crystallinity), P16 (approximately 14% crystallinity) and P24 (approximately 23% crystallinity) were characterized by a lower feed conversion ratio then the control birds (P<0.05) during the starter period. It can thus be concluded that all PHB qualities with crystallinites of 23% or less were able to improve the zootechnical performance of the birds.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art that have the disclosure before them will be able to make modifications and variations therein without departing from the scope of the invention.

### REFERENCES

1. Liu Y, De Schrijver P, Defoirdt T, Van Delsen B, Maignien L, Boon N, Sorgeloos P, Verstraete W and Bossnier P. 2010. PHB-degrading bacteria isolated from the gastrointestinal tract of aquatic animals as protective actors against luminescent vibriosis. FEMS Microbiology Ecology. 74:196-204. SA-12-00588.
2. Defoirdt T, Halet D, Vervaeren H, Boon N, Van de Wiele T, Sorgeloos P, Bossier P and Verstraete W. 2007. The bacterial storage compound poly-beta-hydroxybutyrate protects Artemia franciscana from pathogenic Vibrio campbellii. Environmental Microbiology. 9:445-452. SA-12-00589.
3. Defoirdt T, Boon N, Sorgeloos P, Verstraete W and Bossier P. 2009. Short chain fatty acids and poly-beta-hydroxyalkanoates: (New) biocontrol agents for a sustainable animal production. Biotechnology Advances. 27:680-685. SA-12-01320.
4. Zambell K L, Flitch M D and Fleming S E. 2003. Acetate and butyrate are the major substrates for de novo lipogenesis in rat colonic epithelial cells. Journal of Nutrition. 133:3509-3515. SA-12-00585.
5. Jendrossek D and Handrick R. 2002. Microbial degradation of polyhydroxyalkanoates. Annual Review of Microbiology. 56:403-432. SA-12-02426.
6. Freier T, Kunze C, Nischan C, Kramer S, Sternberg K, Saβ M, Hopt U T and Schmitz K P. 2002. In vitro and in vivo degradation studies for development of a biodegradable patch based on poly(3-hydroxybutyrate). Biomaterials. 23:2649-57. SA-12-02425.
7. Forni D, Wenk C and Bee G. 1999. Digestive utilization of novel biodegradable plastic in growing pigs. Annales de Zootechnie. 48:163-71. SA-12-02421.
8. Forni D, Bee G, Kreuzer M and Wenk C. 1999. Novel biodegradable plastics in sheep nutrition 2. Effects of NaOH pretreatment of poly(3-hydroxybutyrate-co-3-hydroxyvalerate) on in vivo digestibility and on in vitro disappearance (Rusitec). Journal of Animal Physiology and Animal Nutrition. 81:41-50. SA-12-00853.
9. Pluske J R, Williams I H and Aherne F X. 1996. Maintenance of villous height and crypt depth in piglets by providing continuous nutrition after weaning. Animal Science. 62:131-144. SA-12-02484.
10. Shinohe T, Nojiri M, Saito T, Stanislawski T and Jendrossek D. 1996. Determination of the active sites serine of the poly(3-hydroxybutyrate) depolymerases of Pseudomonas lemoignei (PhaZ5) and of Alcaligenes faecalis. FEMS Microbiology Letters. 141:103-109. SA-12-02420.
11. Lobler M, Saβ M, Kunze C, Schmitz K-P and Hopt U T. 2002. Biomaterial patches sutured onto the rat stomach induce a set of genes encoding pancreatic enzymes. Biomaterials. 23:577-583. SA-12-02424
12. Hocking P J, Marchessault R H, Timmins M R, Lenz R W and Fuller R C. 1996. Enzymatic degradation of single crystals of bacterial and synthetic poly(beta-hydroxybutyrate). Macromolecules. 29:2472-2478. SA-12-02446.
13. Abe H and Doi Y. 2002. Side-chain effect of second monomer units on crystalline morphology, thermal properties and enzymatic degradability for random copolyesters of R-3-hydroxybutyric acid with R-3-hydroxyalkanoic acids. Biomacromolecules. 3:133-138. SA-12-02427.
14. Iwata T, Doi Y, Nakayama S, Sasatsuki H and Teramachi S. 1999. Structure and enzymatic degradation of poly(3-hydroxybutyrate) copolymer single crystals with an extracellular poly(3-hydroxybutyrate) depolymerase from Alcaligenes faecalis T1. International Journal of Biological Macromolecules. 25:169-176. SA-12-02422.
15. Kasuya K, Inoue Y, Yamada K and Doi Y. 1995. Kinetics of surface hydrolysis of poly[(R)-3-hydroxybutyrate] film by poly(3-hydroxybutyrate) depolymerase from Alcaligenes faecalis T1. Polymer Degradation and Stability. 48:167-174. SA-12-02419.
16. Bonartseva G, Myshkina V, Nikolaeva D, Rebrov A, Gerasin V and Makhina T. 2002. The biodegradation of poly-β-hydroxybutyrate (PHB) by a model soil community: the effect of cultivation conditions on the degradation rate and the physicochemical characteristics of PHB. Microbiologiya. 71:221-226. SA-12-02428.
17. Qu X-H, Wu Q, Zhang K-Y, Chen G-Q. 2006. In vivo studies of poly(3-hydroxybutyrate-co-3-hydroxyhexanoate) based polymers: biodegradation and tissue reactions. Biomaterials. 27:3540-3548. SA-12-02429.
18. Sudesh K, Abe H and Doi Y. 2000. Synthesis, structure and properties of polyhydroxyalkanoates: biological polyesters. Progress in Polymer Science. 25:1503-1555. SA-12-02423.
19. University Gent. Hydroxybutyrate and poly-hydroxybutyrate as components of animal feed or feed additives. 2010. United States Patent Application US2010/0093860A1.
20. Berg J M, Tymoczko J L and Stryer L. (2006). Biochemistry (6^{th} edition). New York: W.H. Freeman and Company.
21. Sarkar NK. 1972. Ketone body metabolism in chickens. International Journal of Biochemistry. 3:111-116. SA-12-02489.
22. Guilloteau P, Martin L, Eeckhaut V, Ducatelle R, Zabielski R and Van Immerseel F. 2010. From the gut to the peripheral tissues: the multiple effects of butyrate. Nutrition Research Reviews. 23:366-384. SA-11-01426.
23. De Schryver P, Sinha A K, Kunwar P S, Baruah K, Verstraete W, Boon N, De Boeck G and Bossier P. 2009. The bacterial storage compound poly-B-hydroxybutyrate (PHB) increases growth performance and intestinal bacterial range-weighted richness in juvenile European sea bass. Applied Microbiology and Biotechnology. 86:1535-1541. SA-12-02430.
24. Dibner J J and Buttin P. 2002. Use of organic acids as a model to study the impact of gut microflora on nutrition and metabolism. Journal of Applied Poultry Research. 11:453-463. SA-09-0458.
25. Berman H A and Weinstein L. 1971. Antibiotics and nutrition. The American Journal of Clinical Nutrition. 24:260-264. SA-12-02418.
26. Choct M. 2009. Managing gut health through nutrition. British Poultry Science. 50:9-15. SA-10-01957.
27. Abdel-Fattah S A, El-Sanhoury M H, El-Mednay N M and Abdel-Azeem F. 2008. Thyroid activity, some blood constituents, organs morphology and performance of broiler chicks fed supplemental organic acids. International Journal of Poultry Science. 7:215-222. SA-12-02876
28. Adil S, Banday T, Ahmat Bhat G, Salahuddin M, Raquib M and Shanaz S. 2011. Response of broiler chicken to dietary supplementation of organic acids. Journal of Central European Agriculture. 12:498-508. SA-12-02875.
29. Halet D, Defoirdt T, Van Damme P, Vervaeren H, Forrez I, Van de Wiele T, Boon N, Sorgeloos P, Bossier P and Verstraete W. 2007. Poly-β-hydroxybutyrate-accumulating bacteria protect gnotobiotic Artemia franciscana from pathogenic Vibrio campbellii. FEMS Microbiol Ecol 60:363-369.

We claim:

1. A method of improving feed conversion in an animal, comprising the step of feeding the animal an efficacious amount of dispersible and amorphous poly(3-hydroxybutyrate/3-hydroxyvalerate).

2. A method of improving the biodegradability of poly(3-hydroxybutyrate/3-hydroxyvalerate), comprising the step of increasing the dispersiblity and reducing the crystallinity of the poly(3-hydroxybutyrate/3-hydroxyvalerate).

3. A method of reducing the blood glucose level in an animal or a human, comprising the step of feeding the animal or human an efficacious amount of dispersible and amorphous poly(3-hydroxybutyrate/3-hydroxyvalerate).

4. A method of increasing glucagon-like peptide (GLP-1 and GLP-2) production in an animal or a human, comprising the step of feeding the animal or human an efficacious amount of dispersible and amorphous poly(3-hydroxybutyrate/3-hydroxyvalerate).

5. A method of lowering DPP-4 levels production in an animal or a human, comprising the step of feeding the animal or human an efficacious amount of dispersible and amorphous poly(3-hydroxybutyrate/3-hydroxyvalerate).

6. A method of treating conditions such as diabetis, and intestinal diseases such as, but not limited to, inflammatory bowel diseases (IBD), colon cancer, short bowel syndrome (SBS), and chemotherapy-induced apopthosis in an animal or a human, comprising the step of feeding the animal or human an efficacious amount of dispersible and amorphous poly(3-hydroxybutyrate/3-hydroxyvalerate).

7. The methods of claims 1-6 comprising the administration of one or multiple micro-organisms capable of producing dispersible and amorphous poly(3-hydroxybutyrate/3-hydroxyvalerate).

8. The methods of claims 1-6 comprising the administration of purified or intrabacterial poly(3-hydroxybutyrate/3-hydroxyvalerate) in combination with a PBH/PHBV depolymerase or a microorganism capable of producing such depolymerase.
